(19) United States
(12) Patent Application Publication
(10) Pub. No.: US 2007/0066862 A1
(43) Pub. Date: Mar. 22, 2007

(54) Devices and methods for providing cardiac assistance

(76) Inventor: Matthias Vaska, Palo Alto, CA (US)

Correspondence Address: HOEKENDIJK & LYNCH, LLP, P.O. BOX 4787, BURLINGAME, CA, 94011-4787, US

(21) Appl. No.: 11/417,521

(22) Filed: May 3, 2006

Related U.S. Application Data
(60) Provisional application No. 60/678,957, filed on May 6, 2005.

(51) Int. Cl. A61M 1/10

(52) U.S. Cl. 600/16

(57) ABSTRACT

The present invention provides a cardiac assist device which exerts pressure against the epicardial surface to assist the heart in pumping blood. The device may form a pumping space adjacent to an epicardial ventricular surface and/or an epicardial atrial surface. The cardiac assist device may permit the fluid to come into direct contact with the epicardium. The cardiac assist device may include a flexible jacket which at least partially collapses when fluid is withdrawn from the pumping space.

### RELATED APPLICATIONS

This application claims the benefit of U.S. Provisional Application No. 60/678,957 filed May 6, 2005, which is hereby incorporated by reference.

### BACKGROUND OF THE INVENTION

The present invention is related to devices and methods for assisting the heart. In particular, the present invention is related to devices and methods which apply external fluid pressure to the heart to assist the heart in pumping blood. The present invention may be used to assist the heart for any reasons but is particularly useful in treating congestive heart failure. Congestive heart failure is a common and often highly debilitating disease that progressively reduces the ability of the heart to pump blood.

### SUMMARY OF THE INVENTION

The present invention provides various methods and devices for assisting the heart in pumping blood. The present invention provides methods and devices for assisting one or more atria and/or one or more ventricles which may be combined or practiced separately.

In one aspect of the present invention, a cardiac assist device is provided which has a flexible jacket, a fluid delivery system having a pump, and a cardiac monitoring device. The cardiac monitoring device is coupled to the pump so that the pump delivers fluid in a manner which assists the heart. The jacket is positioned between the epicardium and pericardium with the cardiac assist device creating a first pumping space positioned to apply fluid pressure to the epicardium. Fluid is pumped into and out of the first pumping space with the pump in a manner which assists the heart in pumping blood in response to cardiac activity monitored by the cardiac monitoring device. The jacket has an inner surface exposed to fluid in the first pumping space and the jacket is collapsible so that when fluid is pumped out of the first pumping space the jacket at least partially collapses. Although the jacket may be somewhat flexible and compliant, the jacket preferably does not distend significantly when exposed to the pressure forces in the pumping space. Thus, when fluid is delivered into the pumping space the jacket may expand but does not stretch or expand elastically.

In another aspect of the present invention, the fluid is in direct contact with the epicardial surface. An advantage of providing direct fluid contact is that the pressure exerted on the heart may be more uniform compared to prior art solutions which use elastic balloons or membranes to compress the heart. Prior art devices which use an elastic balloon or membrane to squeeze the heart suffer from the problem that the balloons or membranes become relatively rigid structures due to the wall tension created when the balloon or membrane expands. The expanded balloon or membrane will act somewhat like a rigid structure which deforms the heart into a shape dependent upon the expanded shape of the balloon or membrane rather than on the natural shape of the heart at any point in time. For example, U.S. Pat. Nos. 3,455,298 and 5,119,804, to Anstadt disclose an elastomeric liner. One disadvantage which occurs when the liner is inflated and stretched is that a difference in pressure is applied on opposite sides of the liner due to elastic wall tension which develops in the liner. The resulting pressure distribution is not uniform over the surface of the heart and, furthermore, the liner may tend to bulge in the middle so that the heart is deformed into an hour-glass shape. Thus, the elastic liner of the Anstadt patents applies pressure to the heart in a non-uniform manner and also causes the heart to indent unnaturally in its center portion. Because the shape of the heart varies from patient to patient and because the shape is constantly changing as the heart contracts and expands, it is extremely difficult to design such an expandable element that does not deform the heart. Significant deformation of the natural ventricular anatomy should be avoided in order to minimize complications with long term assist.

In one aspect of the present invention, substantially uniform fluid pressure is applied to the exterior surface of the heart with a compliant, flexible sheath. The device may have an encircling attachment and/or seal near the atrioventricular groove (see below) that serves the function of at least partially containing the fluid pressure. This allows the sheath to simply provide a protective function. Because the sheath does not need to contain the fluid pressure, it does not need to support wall tension and therefore may be very thin and flexible. Thus, the sheath may have sufficient flexibility so that the material conforms to the natural outer dimensions of a heart, thereby avoiding significant deformation of the heart.

A seal may be created between the cardiac assist device and the epicardium to prevent fluid from escaping from the pumping space. The seal may encircle the heart such as around the atrioventricular groove (AV groove) and may be positioned closer to the AV groove than to the apex of the heart. The seal may be formed by attaching the cardiac assist device to the heart using an adhesive, gasket, biological adhesion promoting means, sutures and/or piercing elements.

In still another aspect of the present invention, the device is preferably attached to the heart in a manner which permits the heart to displace in a more natural manner at locations inferior to an attachment between the jacket and the heart as compared to prior art devices. Stated another way, the heart is free of attachments to any rigid portion of the cardiac assist device which does not deform due to change in fluid pressure in the pumping space. To this end, the cardiac assist device may also be sealed and/or attached to the pericardium to help anchor the device. The cardiac assist device may also include a supporting element attached to the exterior surface of the pericardium. The supporting element may also be attached to the rest of the cardiac assist device using sutures, staples or another suitable connector which extends through the pericardium. The cardiac assist device may also include a strap which extends around the heart which helps secure the cardiac assist device to the heart.

The cardiac assist device may also be attached to the heart to reduce frictional contact between portions of the cardiac assist device and the heart. To this end, the cardiac assist device may include protective elements attached to the heart such as a sheath or a plurality of elements such as a plurality of independent bands.

In yet another aspect of the present invention, a cardiac assist device is provided which forms a pumping space adjacent at least one atrial epicardial wall. The pumping space may be created in a manner which permits direct fluid contact with the epicardium. The cardiac assist device may also deliver fluid to a second pumping space which exerts pressure on at least one ventricular epicardial wall. The fluid delivery system may be used to pump fluid into the second pumping space when withdrawing fluid from the first pumping space and to pump fluid into the first pumping space when withdrawing fluid from the second pumping space. In another aspect, the pump may be a bidirectional pump, such as a diaphragm pump, which pumps fluid to the first pumping space in one direction and pumps fluid to the second pumping space when pumping in the other direction.

These and other aspects of the present invention are described below in connection with the description of the preferred embodiments.

### BRIEF DESCRIPTION OF DRAWINGS

FIG. 1A shows a cardiac assist device according to the present invention.

FIG. 1B shows the cardiac assist device of FIG. 1A during a different portion of the cardiac cycle.

FIG. 2 is a schematic diagram of the device showing various parts of the device coupled to a control system.

FIG. 3 shows the cardiac assist device.

FIG. 4 is a cross-sectional view of the cardiac assist device.

FIG. 5 is a cross-sectional view of the cardiac assist device mounted to the heart.

FIG. 6 is a partial cross-sectional view of a sheath.

FIG. 7 is a partial cross-sectional view of another sheath.

FIG. 8 is a partial cross-sectional view of still another sheath.

FIG. 9 shows a protective element attached to the heart.

FIG. 10 shows a plurality of protective elements attached to the heart.

FIG. 11 shows still another protective element attached to the heart.

FIG. 12 shows yet another protective element.

FIG. 13 shows a jacket 18 having longitudinal protrusions.

FIG. 14 shows a jacket 18 having circumferential protrusions.

FIG. 15 shows a jacket 18 having a plurality of protrusions.

FIG. 16 shows another cardiac assist device.

FIG. 17 is a cross-sectional view of the cardiac assist device of FIG. 16.

FIG. 18 shows still another cardiac assist device.

FIG. 19 is a cross-sectional view of the cardiac assist device of FIG. 18.

FIG. 20 shows yet another cardiac assist device which uses suction to adhere the device to the heart.

FIG. 21 is a cross-sectional view of the cardiac assist device of FIG. 20.

FIG. 22 shows a docking element attached to the heart.

FIG. 23 shows the rest of the cardiac assist device attached to the docking element.

FIG. 24 is a cross-sectional view showing the interconnection between the docking element and the cardiac assist device.

FIG. 25 shows a fluid distribution element extending around the apex.

FIG. 26 shows a cardiac assist device which has left and right pumping spaces to assist the left and right ventricles independently.

FIG. 27 is a cross-sectional view of a flexible separator extending between the two pumping spaces.

FIG. 28 shows another cardiac assist device.

FIG. 29 shows a fluid distribution ring of the cardiac assist device of FIG. 28.

FIG. 30 shows another cardiac assist device.

FIG. 31 shows a fluid distribution ring of the cardiac assist device of FIG. 30.

### DETAILED DESCRIPTION

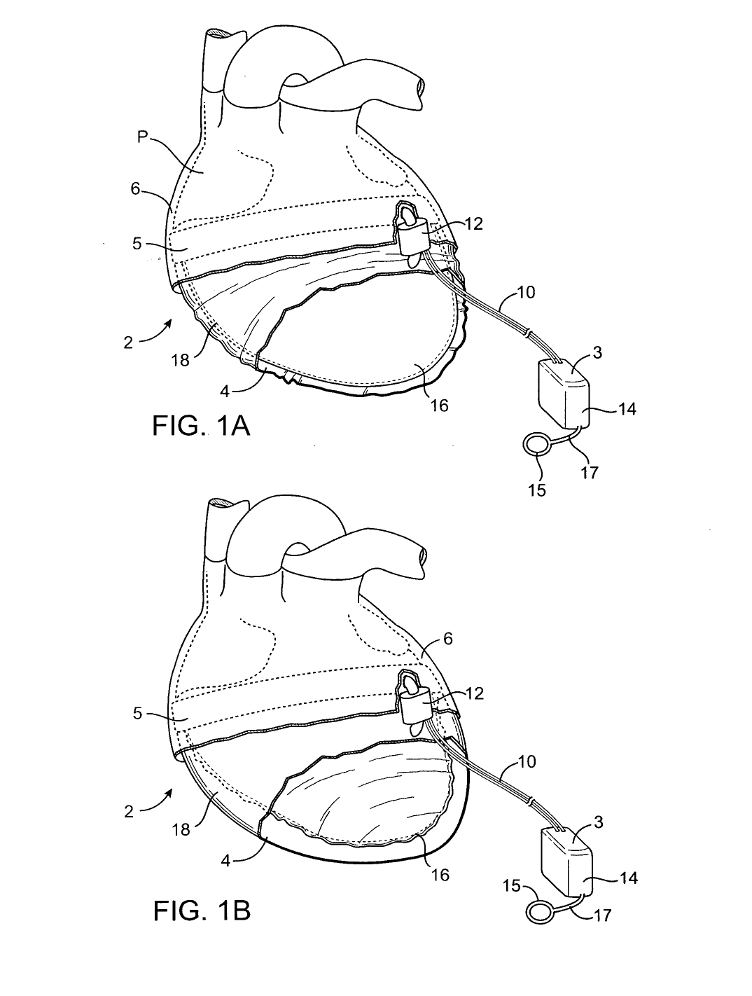

Referring to FIGS. 1-5, a cardiac assist device 2 in accordance with the present invention is shown. The device 2 includes a first pumping space 4 which assists one or both ventricles and a second pumping space 6 which assists one or both atria in pumping blood. Fluid (for example, a gas such as CO2 or a liquid such as saline) is pumped into and out of the pumping spaces 4, 6 so that fluid pressure is exerted on the outside wall of the heart to aid the heart in pumping blood. As will be discussed below, some advantages are found when practicing both atrial and ventricular support, however, the present invention provides aspects for atrial and ventricular support which may be practiced independently.

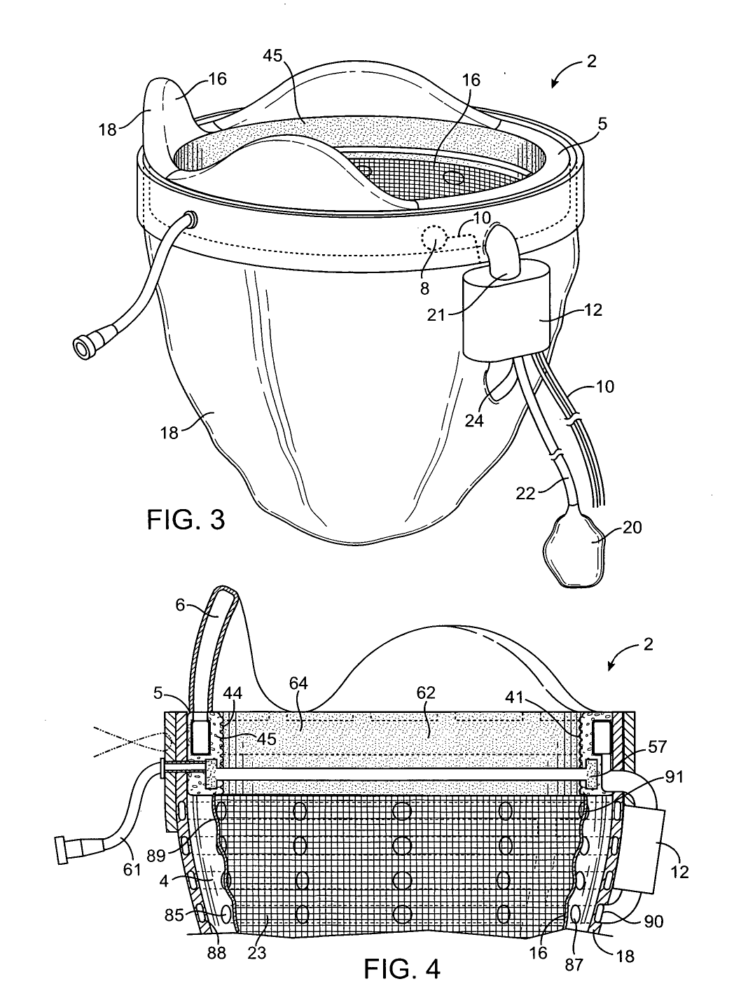

The cardiac assist device 2 includes a cardiac monitoring device 8 with one or more leads 10 attached to the heart to monitor cardiac activity. The cardiac monitoring device 8 is coupled to a pump 12 through a control system 3, which may be integrated with a battery 14, so that the pump 12 delivers and withdraws fluid in a manner which assists the heart. This manner may include synchronous assist with the natural contractions of the heart or an automatic mode when no contractions are detected (e.g. during ventricular fibrillation). The cardiac monitoring device 8 may be any suitable cardiac monitoring device 8 such as a pressure sensor or a sensing electrode as used in pacemakers or defibrillators The cardiac monitoring device 8 is shown exaggerated in FIG. 3 to illustrate the invention and could, of course, be integrated into the same housing as the pump 12 or another part of the cardiac assist device 2.

The battery 14 is used to power the system and may be integrated with the pump 12 or may be implanted at another location. The battery 14 may be recharged through the patient's skin using an induction coil 15 as is known in the art. Although the cardiac assist device 2 may be implanted for long term support, numerous aspects of the present invention may be practiced with the cardiac assist device 2 being a temporary support device. When used as a temporary support device, the pump 12 and power source may be positioned outside the patient.

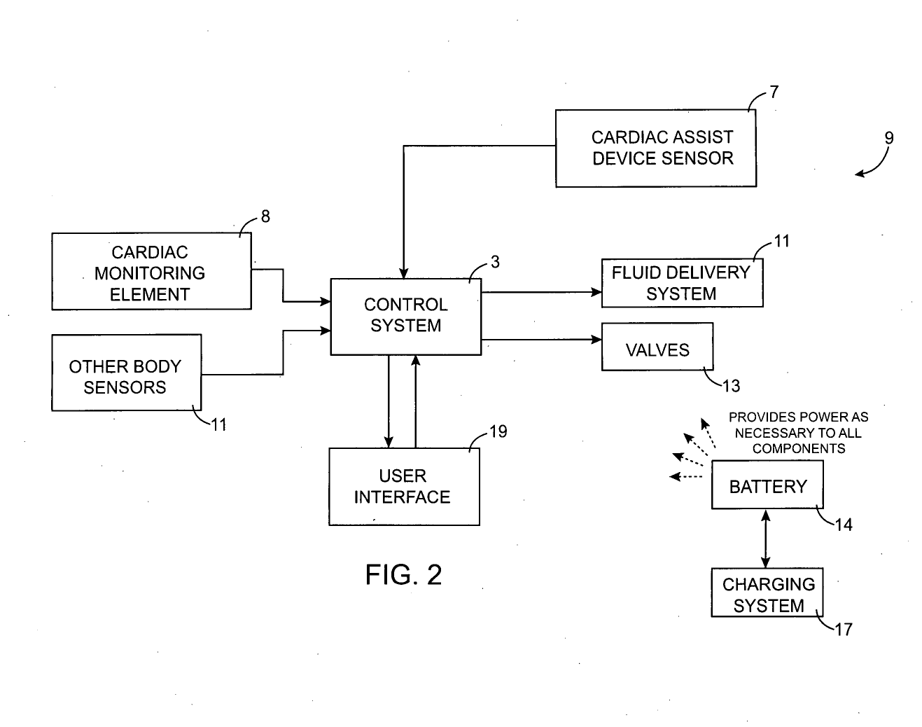

Referring to FIG. 2, a diagram of a system 9 in accordance with the present invention is shown. The system 9 includes the control system 3 which controls the fluid delivery system 11 which includes the pump 12. The control system 3 may receive information from sensors 7 regarding pressure in the pumping spaces 4, 6 and from other sensors 11 regarding other parameters such as temperature, fluid pressures (e.g., of blood or pumping fluid), electrical parameters (e.g., EKG signals or power supply status), optical parameters, fluid flow (e.g., cardiac output or pumping fluid flow), physical forces, acceleration, and motion. The system 3 is also coupled to the cardiac monitoring device 8 and to the pump 12 and any valves 13 which may be needed to direct fluid as desired. The valves 13 may be used to direct fluid between the pumping spaces 4, 6 and/or any other fluid holding element in the system 9. The system 9 may also include a user interface 19 where the user may select an assist level, receive information from a data log or may be notified of one or more alarms. The control system 3 may also receive inputs from the patient or clinician through a wireless or wired user interface 19. The battery 14 is also shown coupled to a charging system 17 which may be the induction coil 15 (see FIG. 1A). The battery 14 is coupled to other parts of the system 9 requiring power such as the pump 12. The control system may also be connected to other devices such as a pacemaker and/or a defibrillator. The control system may also include failsafe features (e.g., pump shutdown and alarm activation) that activate when the sensor input values are not in normally expected operating windows.

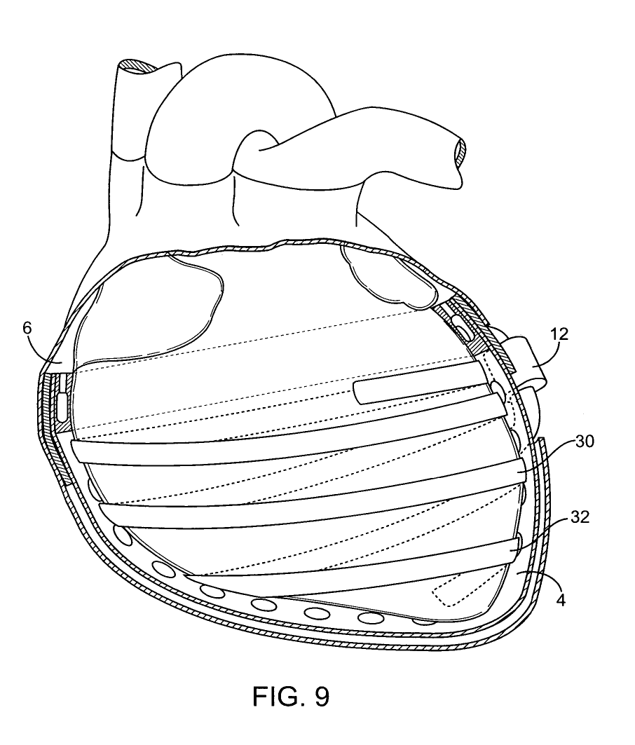

Referring to FIGS. 1A and 1B and to FIGS. 3, 4 and 9, the cardiac assist device 2 is implanted so that the first and second pumping spaces 4, 6 are created between the epicardium and the pericardium. Fluid in the pumping spaces 4, 6 exerts pressure on the outside wall of the heart to assist the heart in pumping blood. The first pumping space 4 assists one or both ventricles by exerting pressure on the epicardial surface of the ventricles. The device has a body 5 to which are attached a sheath 16 and a jacket 18. The first pumping space 4 is formed between the sheath 16 and the jacket 18 which are both positioned between the epicardium and pericardium P. The second pumping space 6 is positioned adjacent to one or both atria along an epicardial surface. The second pumping space 6 may also have the jacket 18 and the sheath 16 (see FIG. 3) which may incorporate the features of any of the jackets 18 or sheaths 16 described herein and those features are expressly incorporated here or the second pumping space 6 may be created in a natural space between the epicardium and pericardium (see FIG. 9) as described further below. The pump space 4 may apply pressure to a substantial portion of the epicardial surface. For example, the pumping space 4 may exert pressure on substantially the entire epicardium inferior to the AV groove, inferior to the attachment or seal between the device 2 and the heart, or at least to all areas on the epicardium nearer to the apex than to the AV groove. The jacket 18 may be made of polyurethane or any other suitable material.

The same fluid may be used to fill and evacuate the first and second pumping spaces 4, 6 so that the fluid is essentially transferred between the two pumping spaces 4, 6. As can be appreciated, the fluid may be directed between the two pumping spaces 4, 6 in a controlled manner which assists one or both atria and also assists one or both ventricles. The fluid may be moved between the two pumping spaces 4, 6 since blood is normally pumped alternately by the ventricles and the atria. The fluid may be pumped between the two pumping spaces 4, 6 in any appropriate manner, however, an advantage of the present invention is that pumping the fluid out of one of the pumping spaces 4, 6 generally means that fluid is being pumped into the other pumping space so that pumping assistance occurs during diastole and systole. Another advantage of this approach is that no fluid compliance chamber or external body vent is required in order to store the fluid when it is pumped out of an assistive pumping space. Full implantability is thus facilitated. The pump 12 may be a reciprocating pump (e.g., diaphragm and piston pumps) which pumps fluid in both directions to each of the pumping spaces 4, 6. A bidirectional pump provides obvious benefits when delivering fluid to the two pumping spaces 4, 6. The pump may, of course, also pump in one direction (e.g., axial and centrifugal pumps) with valving which directs the fluid to the appropriate pumping space 4, 6. Of course, the pumping spaces 4, 6 may be independently filled with the same pump 12 or with two different pumps and even two different fluid sources without departing from the invention.

The jacket 18 may be somewhat flexible so that it may generally conform to the contours of the heart. The outer jacket 18 may be flexible enough to at least partially collapse when the heart contracts. The flexible, compliant outer jacket 18 permits the device 2 to conform not only to the shape of the heart but also facilitates implantation of the device 2 between the epicardium and pericardium. Many prior art devices use a rigid outer shell which may be difficult to fit inside the patient's chest and will not conform to the shape of the heart or to the shape of the space between the pericardium and epicardium. The jacket 18 may be flexible so that the natural motion of the heart is not overly restricted as can occur in some devices described in the prior art. The jacket 18 may be flexible enough so that portions of the jacket 18 may wrinkle, buckle and/or change between convex and concave shapes when the heart is beating. The jacket 18 may also be flexible enough to permit portions of the jacket 18 to collapse and expand when fluid is pumped into and out of the pumping space 4. Although use of a flexible jacket 18 provides various advantages described herein, numerous aspects of the present invention may be practiced with a rigid or semi-rigid jacket 18.

The compliant, flexible outer jacket 18 may also be somewhat safer than prior art devices which use a rigid outer shell since such devices might impede cardiac motion if the pumping element 12 or other critical element fails. A compliant, flexible outer jacket 18 may still permit significant cardiac motion even when a critical element, such as the pump 12, fails. A rigid outer shell, on the other hand, might impede cardiac motion if pressure forces within the rigid shell develop which resist cardiac motion. A rigid shell may also excessively limit natural movement of the heart depending upon the manner in which the device is attached or adhered to the heart Some prior art devices, for example, attach a rigid part of the device to the apex and/or other contracting portion of the heart. As such, it can be appreciated that anchoring a device in this manner may overly limit the natural movement of the heart.

The device 2 encircles the heart and is attached and/or sealed to the heart at or near the AV groove. The manner in which the device 2 is attached and/or sealed to the heart permits the heart to displace in a relatively normal manner. To this end, the device 2 may be free of attachments between the heart and rigid or non-collapsible portions of the device 2. Some prior art devices, for example, use suction to adhere a rigid or non-collapsible portion of device 2 to the apex of the heart. A problem with this method is that inferior portions of the heart, and particularly the apex, are not free to twist and shorten in a natural manner. The device 2 may not have any rigid portion attached to, or even contacting, the heart at locations nearer to the apex than to the AV groove so that at least the inferior portion of the heart, including the apex, may displace in a relatively normal manner. As used herein, a “rigid portion” of the device 2 is any part which does not deform due to a change in fluid pressure in the pumping space 4 or due to forces exerted on it by the beating heart. Stated another way, the device may be coupled to the heart so that the heart is free of attachments to any portion of the device 2 which does not expand and contract at locations closer to the apex than to the AV groove. The heart may, in fact, be free of attachments to rigid or non-collapsing portions inferior to the attachment or seal between the device 2 and the heart which is essentially the area below the AV groove or at least nearer the apex than the AV groove. Of course, the heart may be free of any attachments to the device inferior to the attachment or seal between the device 2 and the heart which is essentially the area below the AV groove or at least the area nearer to the apex than to the AV groove. Thus, the device may be attached to the heart in a manner which permits the inferior portions of the heart to displace in a relatively normal manner as compared to prior art devices which attach a suction cup to the apex of the heart. Use of a suction cup at the apex may overly restrict the natural motion of the heart as explained above.

Although the jacket 18 may be somewhat flexible and compliant, the jacket 18 preferably does not distend significantly when exposed to the pressure forces in the pumping space. Thus, when fluid is delivered into the pumping space the jacket 18 expands but does not expand elastically as suggested in some prior art patents which use balloons. Of course, the outer jacket 18 may be somewhat distensible and elastic but is preferably relatively inelastic and nondistensible. Furthermore, the present invention may be practiced with the jacket 18 being a rigid shell without departing from numerous aspects of the invention. A rigid shell may have the advantage of providing additional assist during diastole by the application of negative fluid pressures.

The jacket may also incorporate features that help prevent the formation of biological adhesions between the jacket and heart when the pumping fluid is in direct contact with the heart. Anti-adhesion agents such as sodium hyaluronic acid, dextran, caboxymethyl cellulose, and fibrinolytic drugs may be applied to or embedded in the inner surface of the jacket for this purpose.

Referring to FIG. 3, the cardiac assist device 2 may also include other features such as a reservoir 20 into which the fluid is delivered if a critical component, such as the pump 12 or cardiac monitoring device 8 fails. A fluid line 22 leads from the pump 12 to the reservoir 20. A relief valve 24 (shown integrated with the pump 12) is positioned to discharge fluid into the reservoir 20 through the line 22 if the pressure in the pumping space 4, 6 departs from an expected operating window. In this manner, the reservoir 20 prevents fluid pressure in the pumping space 4, 6 from impeding cardiac motion should a critical element fail or should a failure condition exist such as an unacceptable pressure. A pressure sensor 21 (also shown integrated with the pump 12) may be used to monitor the fluid pressure in the pumping space 4, 6 during normal operation. If the fluid pressure is outside an acceptable range (high or low) the cardiac assist device 2 may adjust the operating parameters and/or transfer some or all of the fluid to the reservoir 20 as needed. The reservoir 20 may also be used to store fluid that is used to replenish the pumping fluid, or it may be used as a temporary volume that is used to balance or phase the flow between the pumping spaces. The reservoir 20 itself may also be replenishable via a temporary connection through the skin (not shown). Visualization aids such as radiopaque or sonoluminescent elements (not shown) may also be embedded at various locations in the cardiac assist device 2.

Referring to FIGS. 4-8, the sheath 16 may be permeable to fluid so that fluid contacts the epicardial surface. An advantage of providing direct fluid contact is that the pressure exerted on the heart may be more uniform compared to prior art solutions which use elastic balloons or membranes to compress the heart. Of course, direct fluid contact can be also achieved with no sheath at all. Prior art devices which use an elastic balloon or membrane to squeeze the heart suffer from the problem that the balloons or membranes become relatively rigid structures due to the wall tension created when the balloon or membrane expands. The expanded balloon or membrane will act somewhat like a rigid structure which deforms the heart into a shape dependent upon the expanded shape of the balloon or membrane rather than on the natural shape of the heart at any point in time. Because the shape of the heart varies from patient to patient and because the shape is constantly changing as the heart contracts and expands, it is extremely difficult to design such an expandable element that does not deform the heart. The sheath 16 may be made of any suitable material such as polyurethane or silicone. The sheath 16 may be a woven or braided structure 23 with a relatively open mesh as shown in FIG. 4. The sheath 16 may also be formed from strips of material 25 as shown in FIG. 8 or may have a wrinkled surface 26 as shown in FIG. 6. The sheath 16 may also be impermeable to fluid so that the pumping space 4, 6 is closed and does not permit fluid to contact the heart without departing from numerous aspects of the invention.

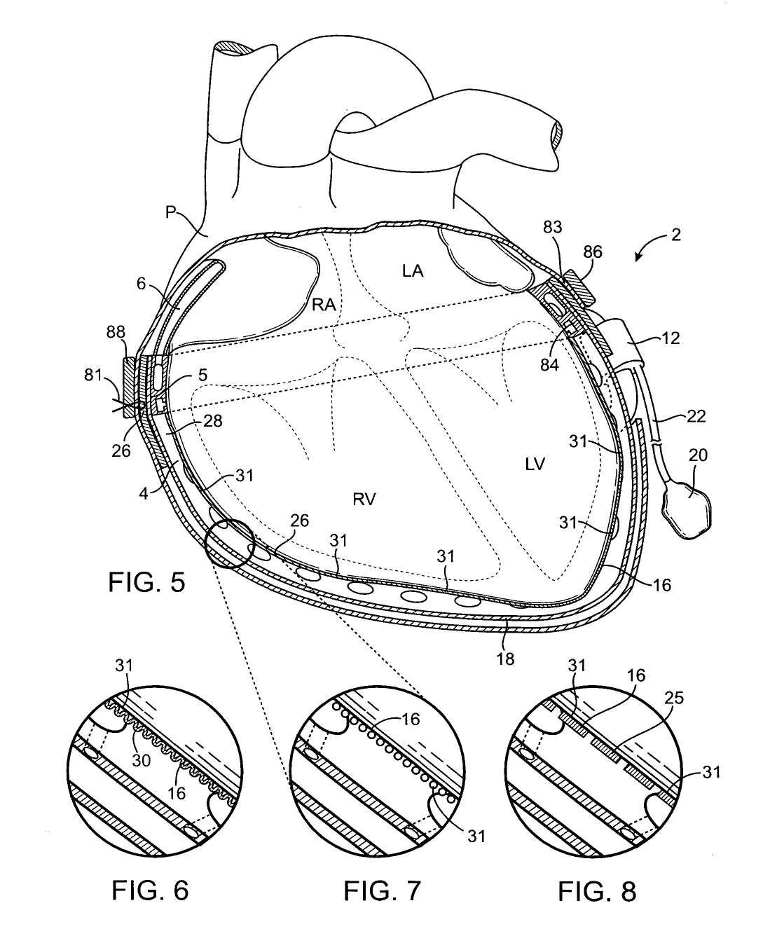

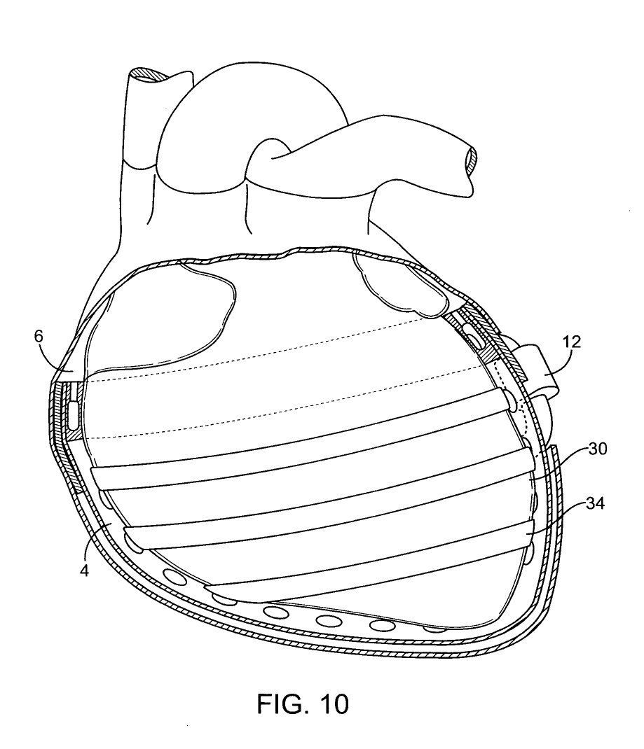

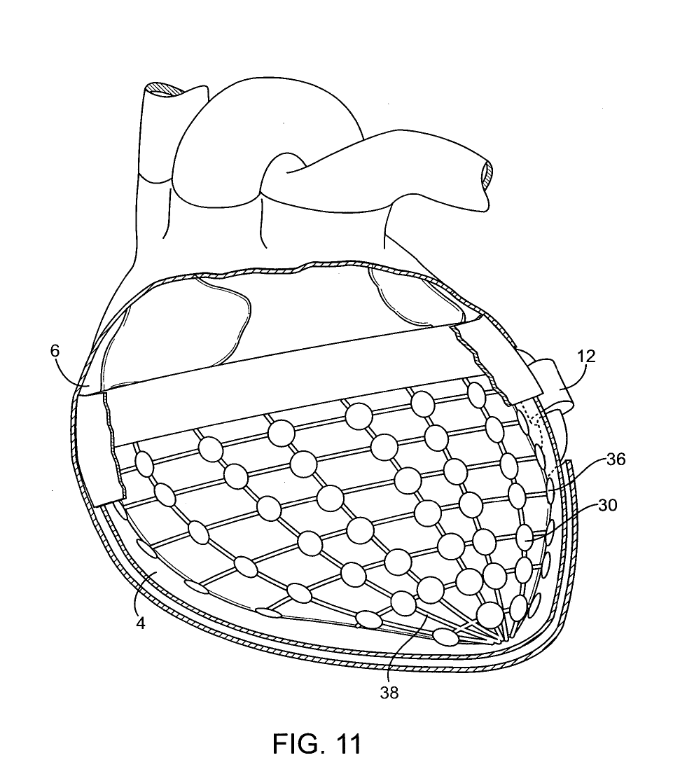

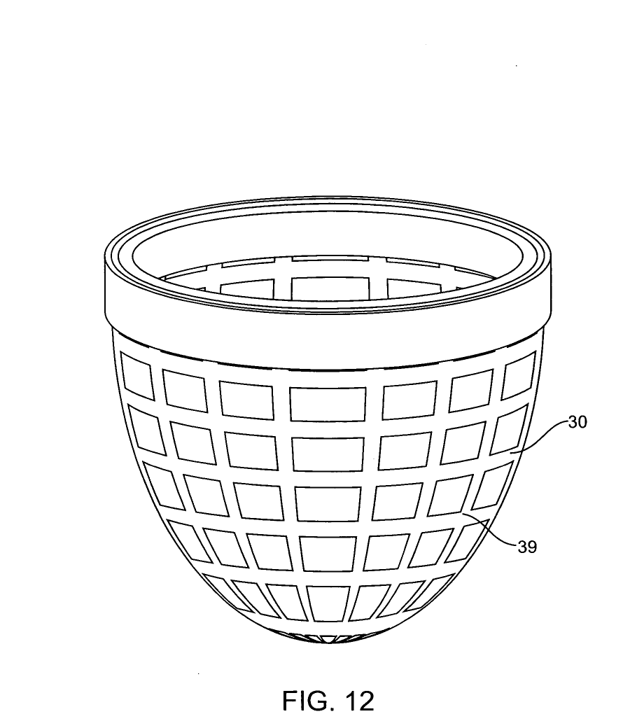

The sheath 16 acts as a protective element 30 for the heart in that the sheath 16 may reduce friction between the jacket 18 and portions of the heart covered by the sheath 16. The sheath 16 may also be attached to the heart to reduce sliding contact between the heart and the cardiac assist device 2. In addition, attaching an impermeable sheath to the heart also prevents it from pulling away from the heart if negative fluid pressure is applied. For example, an adhesive 26 (e.g., adhesives formulated with cyanoacrylate, polyethylene glycol, albumin, glutaraldehyde, or fibrin), biological adhesion promoting means, vacuum, or piercing elements such as sutures 28 may be used to attach the sheath to the heart as shown in FIG. 5. The protective element 30, such as the sheath 16, may be attached to the heart at a plurality of locations 31 as shown in FIGS. 5-8 using any of the methods or devices described herein. The cardiac assist device 2 may also include one or more of the protective elements 30, such as the sheath 16, attached directly to the heart. The protective element 30 may be a continuous strip 32 of material as shown in FIG. 9 or a number of independent strips 34 which are attached to the heart as shown in FIG. 10. Referring to FIG. 11, another protective element 30 is shown which applies a modest compressive force which attaches the element 30 to the heart. The protective element 30 has a plurality of projections 36 which are attached to one another with interconnecting elements 38. Referring to FIG. 12, the protective element 30 may be a web-like structure 39 formed from a suitable flexible material. The protective elements 30, including the sheath 16, may be designed to intimately conform to the heart as it beats regardless of fluid pressure, unlike the balloon and membrane elements used in some prior art devices. Because the protective elements need not stretch with application of fluid pressure, the protective elements may be extremely flexible, thin, and somewhat oversized thereby reducing the need for precise sizing in order to conform to the highly variable anatomy of the heart. Thin and flexible protective elements will impede heart motion the least and allow for better transmission of fluid pressure. Any of the protective elements 30, including the sheath 16, may be used with any of the devices described herein and such use is expressly incorporated.

The sheath 16 may be sized and configured so that the sheath 16 does not expand elastically when fluid is pumped into the pumping space 4 like the balloons and elastic membranes described in some prior art devices. The balloons and membranes described in the prior art expand elastically and develop wall tension which makes the balloons relatively rigid. As such, the balloons do not permit the heart to displace naturally and will tend to force the heart to conform somewhat to the inflated shape of the balloon. An elastic balloon or membrane will also begin to resist introduction of fluid into the pumping space due to the wall tension developed when the balloon is inflated. The sheath 16 of the present invention does not expand elastically when the pumping space 4 is filled and, in fact, portions of the sheath may wrinkle, collapse and/or buckle rather than stretch elastically when fluid is delivered into the pumping space 4. As such, the sheath 16 will also not resist the introduction of fluid into the pumping space 4 and will not develop wall tension as occurs when using an elastic balloon or membrane. The sheath 16 does not need to resist introduction of fluid into the pumping space because the encircling attachment and/or seal of the cardiac assist device 2 provides this function. The sheath 16 may therefore be very thin and compliant, thereby minimizing any deformation of or trauma to the heart. The sheath 16 may also be free of any attachments to rigid or non-collapsing portions of the device at all locations closer to the apex than to the AV groove. Of course, numerous aspects of the present invention may be practiced without the sheath 16 or with the sheath 16 contacting or being attached to rigid or non-collapsing portions of the device 2.

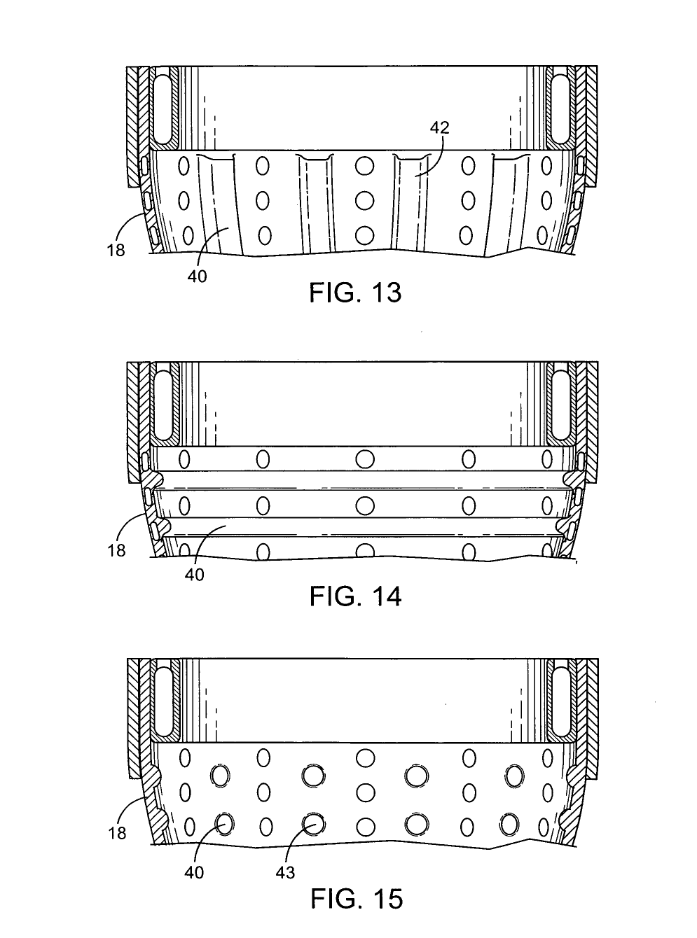

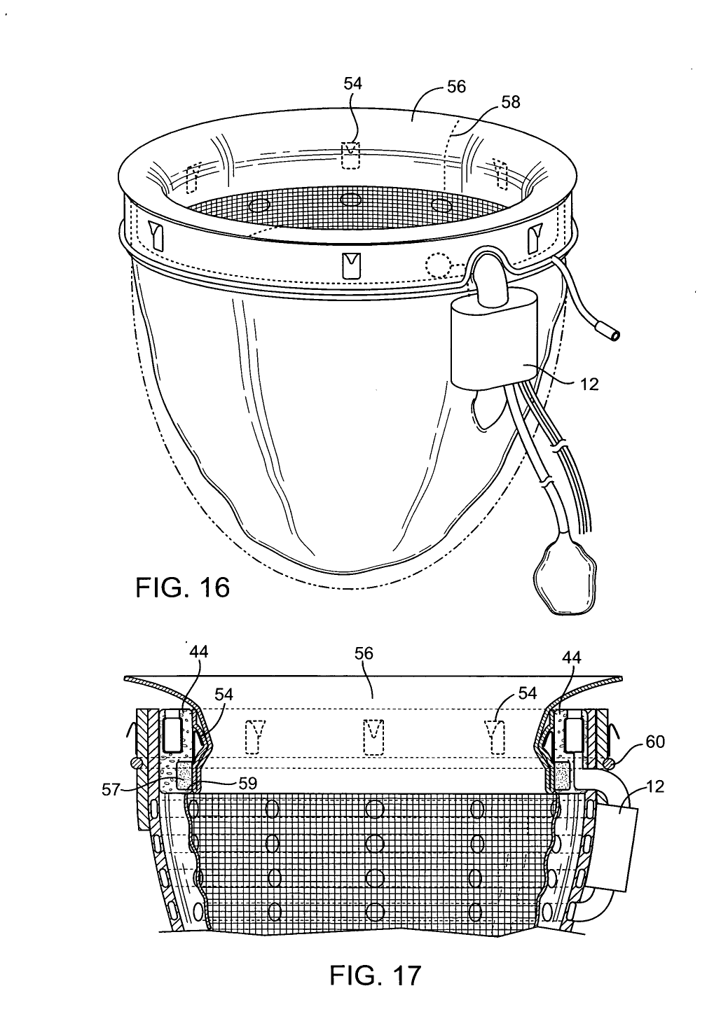

Referring to FIGS. 13-15, the jacket 18 may also have one or more protrusions 40 which help to maintain separation between the jacket 18 and the sheath 16 or between the jacket 18 and the epicardium when no sheath 16 is present. Separation between the jacket 18 and the epicardium or sheath 16 may help to ensure distribution of fluid throughout the jacket 18, may help prevent blockage of fluid ports, and may also reduce contact with the epicardium. The protrusions 40 may be one or more ribs 42 which extend toward the apex as shown in FIG. 13 or which extend circumferentially around the heart as shown in FIG. 14. The protrusions 40 may also simply be raised dimples 43 as shown in FIG. 15. Any of the protrusions 40 may be used with the jackets 18 described herein and such use is expressly incorporated.

When permitting the fluid to come into direct contact with the heart as mentioned above, the cardiac assist device 2 is attached and sealed to the heart to create the pumping spaces 4, 6. Of course, a complete seal may not be necessary but a substantially fluid tight seal is desired. The seal may encircle the heart and may encircle the heart nearer to the AV groove than to the apex so that a substantial portion of the ventricles are exposed when creating the pumping space 4 which assists the ventricles. The seal may encircle the heart at or near the AV groove when creating the pumping space 6 which assist the atria. FIG. 5 shows the seal positioned at the AV groove which seals both pumping spaces 4, 6.

Referring again to FIGS. 3 and 4, the body 5 has a contact member 44 which may be somewhat flexible and resilient. The contact member 44 has a contact surface 41 on an inner side which encircles the heart. The contact member 44 may be formed by a compressible gasket 45 made of closed-cell foam which is impermeable to the fluid. It may also contain a rigid, semi-rigid or malleable element (not shown) in order to provide a predetermined or adjustable shape. The cardiac assist device 2 may be adhered to the heart using an adhesive 57. The adhesive 57 may also be delivered through a delivery lumen 61, a portion of which may be removable. Referring now to FIG. 17, the contact member 44 may also have piercing elements 54 which pierce the epicardium to secure the cardiac assist device 2 to the heart. The piercing elements 54 may be covered during introduction by a peel-away guard 56. The guard 56 has a perforated section 58 to permit the guard 56 to-be removed once the cardiac assist device 2 is positioned around the heart. The guard 56 may also be used to expose the adhesive 57 stored in an annular recess 59 in the contact member 44.

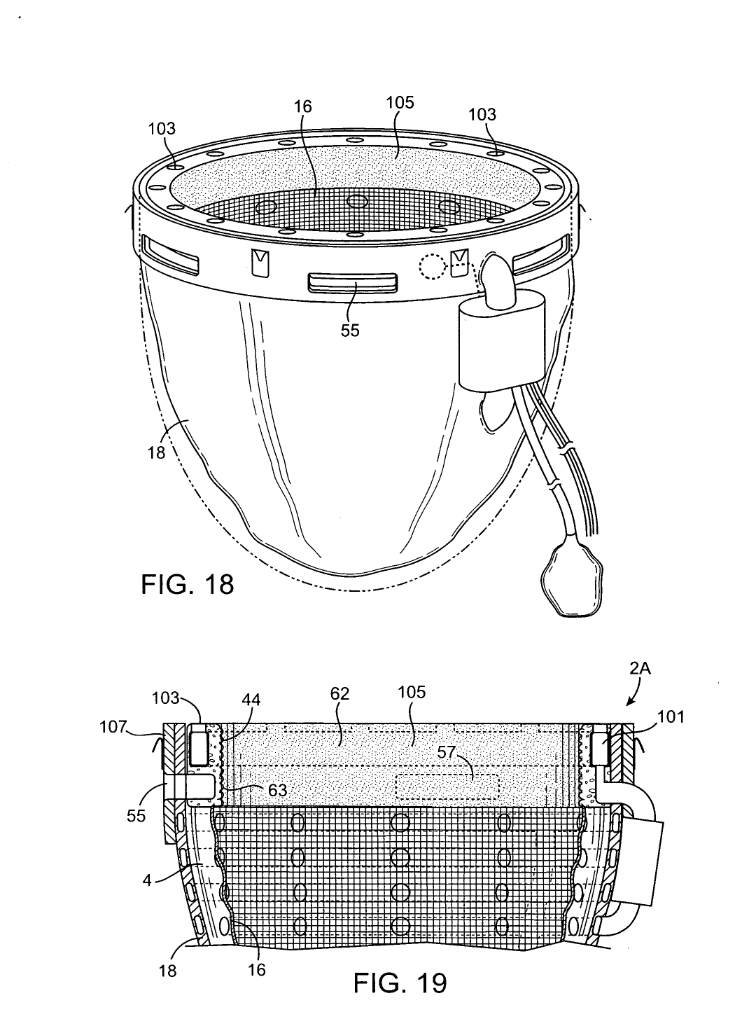

Referring to FIG. 18, the cardiac assist device 2 may also have windows 55 that are configured to allow piercing elements to pass therethrough when attaching the device to the heart. The piercing elements may be staples or the like or may simply be a needle which delivers suture. The device 2 has a band of material 63 integrated with the contact member 44 which is penetrated by the piercing elements to attach the device 2 to the heart.

The cardiac assist device 2 may: also be attached and sealed to the heart by promoting the formation of biological adhesions between the cardiac assist device 2 and the heart. The contact member 44 may also have a rough or porous surface 62 along the contact surface 41 which causes localized abrasion and thus promotes the formation of biological adhesions between the cardiac assist device 2 and the heart as shown in FIGS. 4 and 19. Other methods of creating biological adhesions between the cardiac assist device 2 and heart may also be used such as use of a desiccant or a chemical sclerosing agent 64, such as talc, iodopovidone or tetracycline hydrochloride applied to the cardiac assist device 2. A porous tissue contact surface may also allow ingrowth of the biological adhesions, further strengthening the attachment. As used herein, the term adhering shall mean bonding the device to tissue using an adhesive, sealant, vacuum, or biological adhesion-promoting means such as glue, a sclerosing agent, a desiccant, means of producing mechanical or thermal injury (e.g., rough or porous tissue contact surface, radiofrequency heating) or any other means which causes tissue to bond to the device or to another tissue layer.

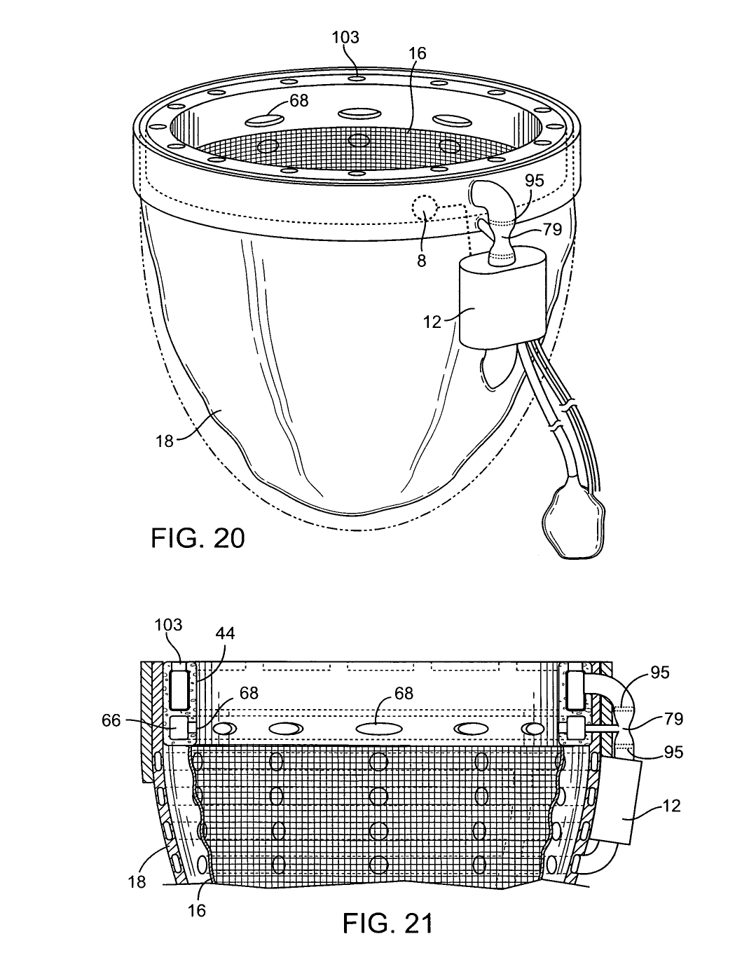

Referring to FIGS. 20 and 21, still another method of adhering the cardiac assist device 2 to the heart is shown wherein the same or similar reference numbers refer to the same or similar structure. The contact member 44 has a vacuum lumen 66 with suction ports 68 to adhere the cardiac assist device 2 to the heart. Suction may be created in any suitable manner and FIG. 20 and 21 show the pump 12 coupled to the vacuum lumen 66 via a venturi 79 to create suction. An advantage of using the venturi 79 to create suction is that a separate vacuum pump is not required. The same pump that pumps fluid into and out of the pumping spaces may drive the venturi. Any fluid that is withdrawn through the vacuum lumen 66 is directed back into the pumping circuit. By minimizing the number and size of fluid delivery components implantability of the system is facilitated.

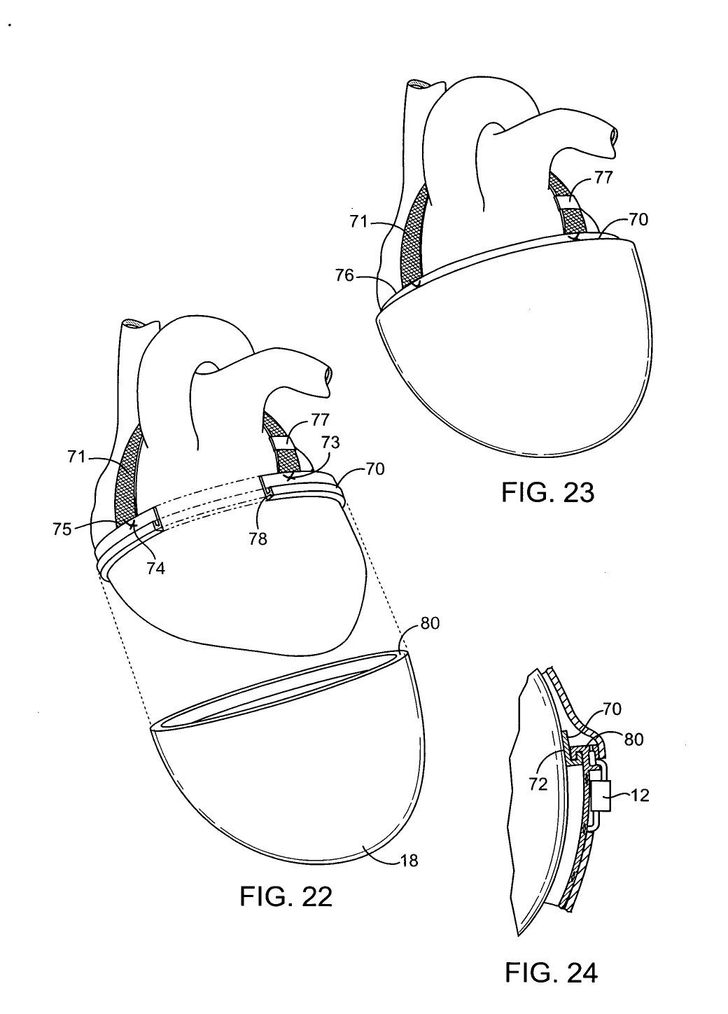

Referring to FIGS. 22-24, still another method of attaching the cardiac assist device 2 to the heart is shown. A docking element 70 is attached to the heart in any suitable manner such as with adhesive 72 and sutures 74. The docking element 70 may be a ring 76 which extends around a circumference of the heart and is attached to the heart near the AV groove. The docking element 70 has a groove 78 which mates with a support ring 80 attached to the jacket 18. The support ring 80 and docking element 70 having each have a groove 82 which mates with the other. The support ring 80 and the docking element 70 may, of course, be coupled together in any other suitable manner. The docking element 70 and support ring 80 will also form a substantially fluid tight seal as necessary such as when the fluid is in direct contact with the heart. The pericardium is attached, and sealed if necessary, to the support ring 80 or another part of the cardiac assist device 2. The docking element 70 may also be secured to the heart with a strap 71 that extends around a vessel in a space between the epicardium and pericardium such as through the transverse sinus. The strap 71 has a first portion 73 and a second portion 75 which are both attached to the docking element 70. The strap 71 includes any suitable releasable attachment 77 along the strap, such as a buckle, so that the strap 71 may be wrapped around a vessel and then closed to secure the device 2. The strap 71 may be used with any of the other devices described herein and such use is expressly incorporated here.

The docking element 70 may come in various sizes with the size being selected based on the geometry of the heart. For example, the docking element 70 may be selected based on the circumference of the heart around the AV groove. The cardiac assist device 2 may also come in various sizes based upon other geometrical considerations such as the distance between the AV groove and the apex of the heart or based on the overall size and geometry of the heart. The docking element 70 may be used with any of the cardiac assist devices 2 described herein.

The cardiac assist device 2 may also be attached to the pericardium to help resist fluid pressure forces which act to potentially dislodge the device. In particular, pressure forces on the cardiac assist device 2 may tend to push the cardiac assist device 2 toward the apex of the heart when the cardiac assist device 2 is positioned around the heart. The cardiac assist device 2 may be attached to the pericardium in any suitable manner. For example, the cardiac assist device 2 may be glued, sutured, clipped, hooked (FIG. 19), suctioned or stapled to the pericardium.

Referring again to FIG. 5, the device 2 may also be sealed to the pericardium. The body 5 has a contact surface 83 on an outer side which may form a seal with the pericardium to seal one or both of the pumping spaces 4, 6. The device 2 may be sealed to the pericardium using any suitable device or method including those described in relation to sealing the device 2 to the heart which are expressly incorporated here. For example, an adhesive 84 and/or sutures 81 may be used to bond the cardiac assist device 2 to the pericardium. An outer member 86, such as a ring 88, may also be used to stabilize the cardiac assist device 2 and secure the cardiac assist device 2 to the pericardium. The outer member 86 may be mechanically attached to the cardiac assist device 2 through the pericardium using sutures, staples or the like and/or with the adhesive 84. The outer member 86 may also be used to compress the pericardium between the outer member 86 and the cardiac assist device 2 in order to facilitate sealing and attaching of the cardiac assist device 2 to the pericardium. Referring to FIG. 17, RF energy may be delivered to a wire 60 which contacts the pericardium. When RF energy is delivered to the wire 60, the damage to the pericardium stimulates a healing response which creates biological adhesions between the cardiac assist device 2 and pericardium. Application of other types of energy transfer such as cryogenic, direct heat, microwave, or laser can also be used to stimulate a healing response. The attachment to the pericardium may be around a circumference of the heart and radially outward from the attachment between the cardiac assist device 2 and the epicardium. Various methods and devices for attaching and/or sealing the cardiac assist device 2 to the heart and to the pericardium are described herein. These methods may be applied interchangeably to either the epicardial or pericardial attachments and/or seals of any of the embodiments described herein and such combinations are expressly incorporated herein. For example, the docking element 70 may be used with the device of FIGS. 3 and 4. Thus any combination of adhesive agents, piercing elements, biological adhesion-promoting means, and vacuum may be used for any or all of the epicardial and pericardial attachments and seals.

The cardiac assist device 2 is attached to the heart near the atrioventricular groove. An advantage of attaching the cardiac assist device 2 to the pericardium and/or epicardium at or near the AV groove is that the AV groove is a relatively stable area and that displacements of the heart below the AV groove are less affected than prior art devices which are attached near the apex of the heart. The present invention permits the heart to displace in a more natural manner compared to many prior art devices because the present invention avoids fixation to areas of high relative cardiac motion. Fixation at the AV groove also allows fluid to exert pressure more evenly over the entire ventricular surfaces and allows the aforementioned protective elements and sheaths to fully enclose and protect the ventricles. In addition, fixation at the AV groove can help to contain the fluid pressure, thereby allowing the aforementioned impermeable sheath 16 to be particularly thin and conforming to the natural anatomy. Many prior art devices attach or adhere a rigid or non-collapsible portion of the device at or near the apex. As such, these devices tend to restrict cardiac motion and, in particular, will limit apical retraction or shortening and will also limit twisting of the heart particularly at the apex. In one aspect of the present invention, the apex is free of attachments to any non-collapsible portion of the cardiac assist device 2. Stated another way, the heart is free of attachments to non-collapsible portions of the cardiac assist device 2 inferior to the seal or attachment between the cardiac assist device 2 and the heart or between the cardiac assist device 2 and the pericardium. Collapsible portion as used herein shall mean any part of the cardiac assist device 2 which collapses, deforms or otherwise changes shape due to pressure or mechanical forces imposed by the beating heart or the fluid in the pumping spaces 4, 6.

Referring again to FIG. 4, the pump 12 has a first fluid outlet portion 85 and a second fluid outlet portion 87 for delivering fluid to and withdrawing fluid from the first and second pumping spaces 4, 6. Each fluid outlet portion 85, 87 is coupled to a fluid channel 88, 90 which delivers the fluid to the pumping spaces 4, 6. The distance between the first and second fluid outlet portions 85, 87 as measured along the outer surface of the heart or inner surface of the jacket 18 is at least 5 centimeters so that fluid is distributed throughout the pumping space 4. For example, the first and second outlet portions 85, 87 may be positioned over the left and right ventricular free walls, respectively. The fluid delivery system may also have a third fluid outlet portion 89 and a fourth fluid outlet portion 91 with the surface area of the outer surface of the heart or the surface area of the jacket 18 spanned by the four ports 85, 87, 89, 91 being at least 20 square centimeters. The fluid outlet portions may be coupled to the jacket 18 or to any other part of the device 2 such as the sheath 16 and are positioned directly opposite the epicardium when the device 2 is placed on the heart so as to minimize the pressure gradient in the pumping space 4 during fluid delivery. The fluid outlet ports 85, 87 may be either discrete fluid outlets or ports or a continuous elongated outlet (such as a spiral slot) or any combination of discrete ports and continuous outlets. The fluid outlet portions may be distributed so that two of the portions are closer to the apex than to the AV groove and two of the outlet portions distribute fluid to each of the left and right ventricular walls. Of course, it can be appreciated that by positioning the outlet portions 85, 87 on diametrically opposing sides of the body 5 that the outlet portions 85, 87 will naturally be positioned to distribute fluid to the left and right ventricles. The fluid outlet portions 85, 87 may also distribute fluid to the same pumping space 4 so that the fluid outlet portions are essentially fluidly coupled to one another through the pumping space 4.

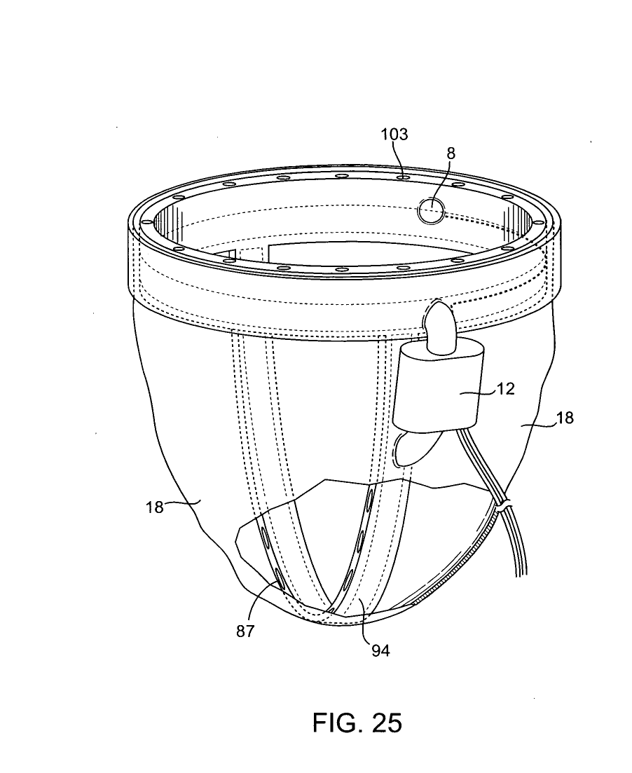

The distribution of fluid outlet portions 85, 87, 89, 91 ensures that fluid may be delivered and removed from all parts of the pumping space 4 as necessary. The distribution of the fluid portions may be particularly helpful when the jacket 18 and/or sheath 16 are somewhat compliant as described herein. The distribution of fluid outlet portions enables a more uniform pressure distribution, minimizes clogging, and minimizes the risk that parts of the pumping space 4 are cut off from fluid if the jacket 18 and/or sheath 16 is adhered or otherwise blocked by the pericardium, epicardium, jacket 18 or sheath 16. Referring to FIG. 25, a fluid distribution element 94 may also extend around the apex of the heart to distribute fluid to both sides of the pumping space 4. The fluid may also be directed through one or more filters to remove material from the fluid. The filters 95 (see FIG. 21) may be particularly helpful when the fluid is in direct contact with the heart to prevent a buildup of biological material in the fluid. The fluid may also include additives such as anti-adhesion agents (e.g., sodium hyaluronic acid, dextran, caboxymethyl cellulose, and fibrinolytic drugs), anti-bacterial agents, lubricants, or anticoagulant agents such as heparin.

Referring to FIGS. 9 and 19, another cardiac assist device 2A is shown which assists both ventricular pumping and atrial pumping wherein the same or similar reference numbers refer to the same or similar structure. FIG. 9 omits some features of the device 2A for clarity. A first pumping space 4 is created to assist one or more ventricles and a second pumping space 6 is created to assist one or more of the atria. The cardiac assist device 2A forms a seal with the heart and pericardium around a circumferential portion of the heart such as near the AV groove. As used herein, the term circumferential shall be construed to include any closed path that encircles the heart. The cardiac assist device 2A may be sealed and attached to the heart and to the pericardium in any manner described herein. For example, the cardiac assist device 2A may be attached or adhered to the heart and/or pericardium using an adhesive or the piercing elements as shown or described herein.

The second pumping space 6 is created between the epicardium and pericardium outside one or both atria. The pump 12 delivers fluid to a channel 101 having ports 103 (FIG. 18) which direct the fluid into a natural space between the epicardium and pericardium. As such, little or no foreign material may be necessary in the second pumping space 6. Of course, the cardiac assist device 2 may include the jacket 18, sheath 16 and/or protective elements for use in connection with the second pumping space 6 without departing from numerous aspects of the invention and such features are expressly incorporated here. The cardiac assist device 2A has a first sealing surface 105 on an inner side which forms a seal with the epicardium and a second sealing surface 107 which forms a seal with the pericardium on an outer side. Both sealing surfaces 105, 107 are continuous and encircle the heart to form the second pumping space 6 superior to the seal.

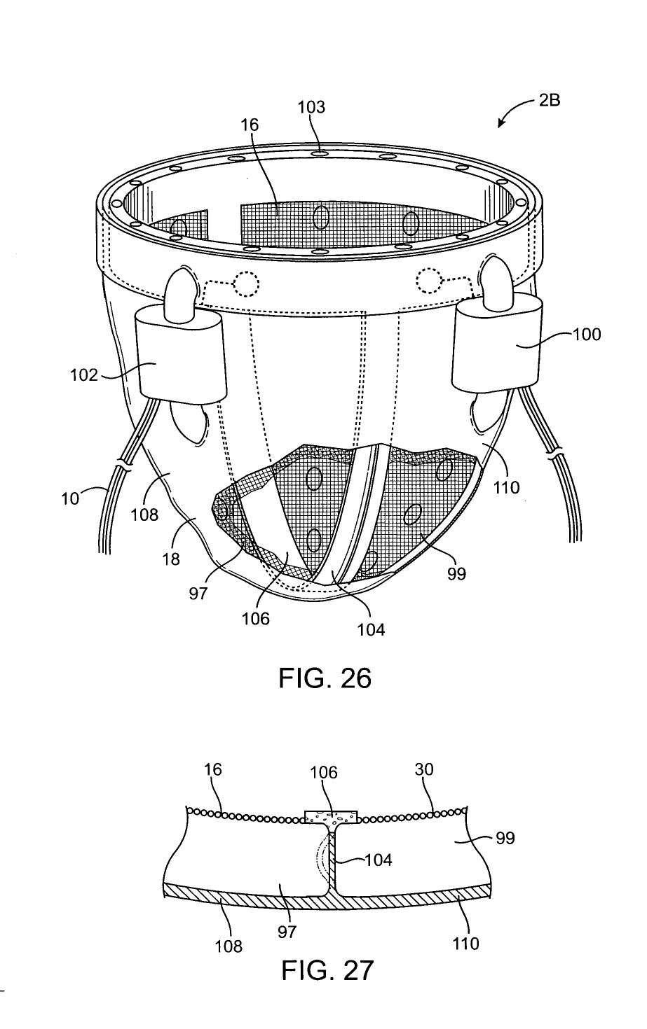

Referring now to FIGS. 26 and 27, still another cardiac assist device 2B is shown. The cardiac assist device 2B has separate pumping 'spaces 97,99 one for each ventricle. Two pumps 100, 102 are provided for separately pumping fluid into the pumping spaces 97, 99 to independently assist the left and right ventricles. Of course, one pump may also be used with appropriate diversion of fluid flow between the two pumping spaces 97, 99. A separator 104 extends along the jacket 18 and has a sealing portion 106 which is attached to the epicardium to separate the left and right pumping spaces 97, 99. The separator 104 may be a thin, flexible, compliant, impermeable material which is adhered, sutured or otherwise attached to the heart. As mentioned above, it is undesirable to restrict the natural motion of the heart and the separator 104 is designed to be flexible and compliant enough to permit the apex to twist and shorten in a relatively natural manner. The separator 104 may also be elastic or otherwise extensible in order to accommodate various distances between the heart and jacket while still maintaining a seal between the pumping spaces.

The separator 104 delineates a first jacket portion 108 and a second jacket 110 portion which form the left and right pumping spaces respectively. The cardiac assist device 2B may also include the protective elements 30 and/or the sheath 16 which may be permeable or impermeable. When permitting the fluid to come into direct contact with the heart, the jacket 108 and separator 104 form a seal with the outer wall of the heart. The jacket 108 and separator 104 may form a seal with the epicardium in any manner described herein or in any other suitable manner. When using an impermeable sheath 16 the separator 104 and jacket 18 will not have to form a seal with the surface of the heart. Of course, the separator 104 may still be attached or adhered to the heart or pericardium to help maintain its position.

Referring to FIGS. 9, 26 and 27, thee second pumping space 6 for the cardiac assist device 2B is created in a natural space between the epicardium and pericardium similar to the second pumping space 6 shown in FIG. 9. The second pumping space 6 is formed by creating a seal between the cardiac assist device 2B and the heart and between the cardiac assist device 2B and the pericardium. The seal between the cardiac assist device 2B and the heart or between the cardiac assist device 2B and the pericardium may be formed in any manner described herein. For example, the adhesive, the outer member 86 (FIG. 5) and/or suction (FIG. 21) may be used to secure the cardiac assist device 2B to the heart and/or pericardium. The separate left and right ventricular pumping arrangement described above may be used with any of the devices described herein and such use is expressly incorporated.

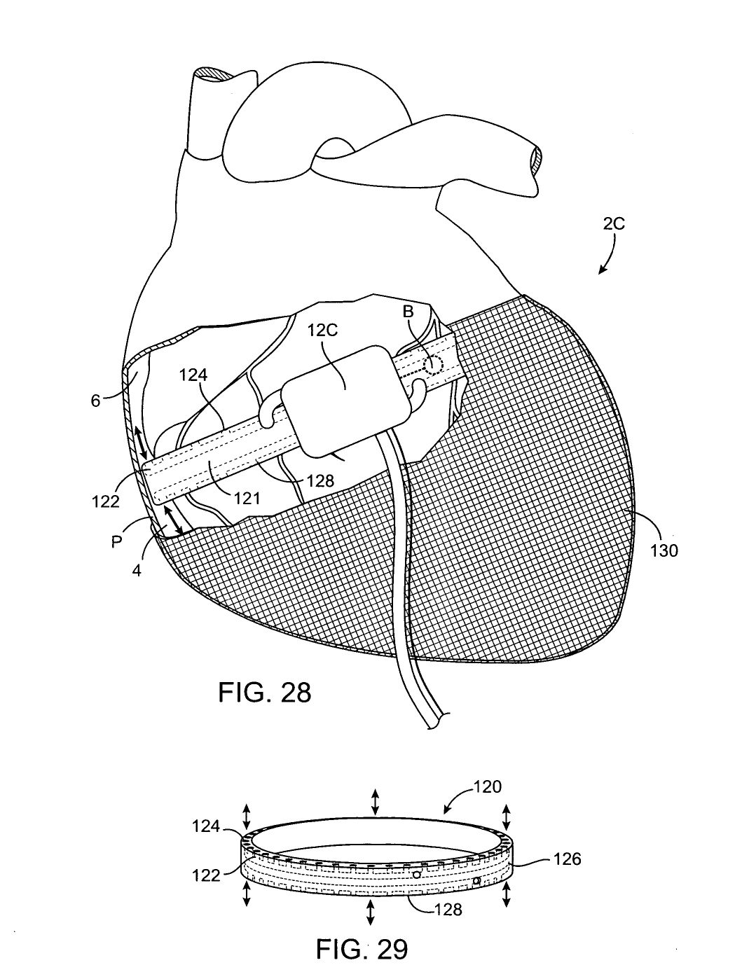

Referring to FIGS. 28 and 29, still another cardiac assist device 2C is shown wherein the same or similar reference numbers refer to the same or similar structure. The cardiac assist device 2C includes pump 12C which pumps a fluid into a first pumping space 4 which assists one or both ventricles and a second pumping space 6 which assists one or both atria. Both the first and second pumping spaces 4, 6 are created in natural spaces between the epicardium and pericardium thereby minimizing the amount of foreign material in contact with the heart. Of course, the jacket 18, sheath 16 and/or protective elements 30 as described herein may also be used in connection with the first and second pumping spaces 4, 6 without departing from the scope of the invention.

The pump 12C is coupled to body 121 which may be ring shaped as shown in FIG. 29. The body 121 includes a fluid distribution element 120 which has a first channel 122 and a first set of openings 124 which deliver fluid to the second pumping space 6 and a second channel 126 and a second set of openings 128 which deliver fluid to the first pumping space 4. The cardiac assist device 2C is attached and sealed to the heart and to the pericardium to separate the first and second pumping spaces 4, 6. Of course, a complete seal may not be necessary but a substantially fluid tight seal is desired. The cardiac assist device 2C may be sealed with the epicardium and/or pericardium in any suitable manner such as those described herein which are expressly incorporated here. For example, the adhesive may be applied to both sides of the distribution element 120 to form a seal with the heart and the pericardium.

A pericardial support member 130 is positioned outside the pericardium to support the pericardium from distending due to pressure created in the first pumping space 4. The pericardial support 130 member may be a mesh-like material which is relatively non-distensible. The pericardial support member 130 may be mechanically attached or adhered to the pericardium with an adhesive, sutures or the like.

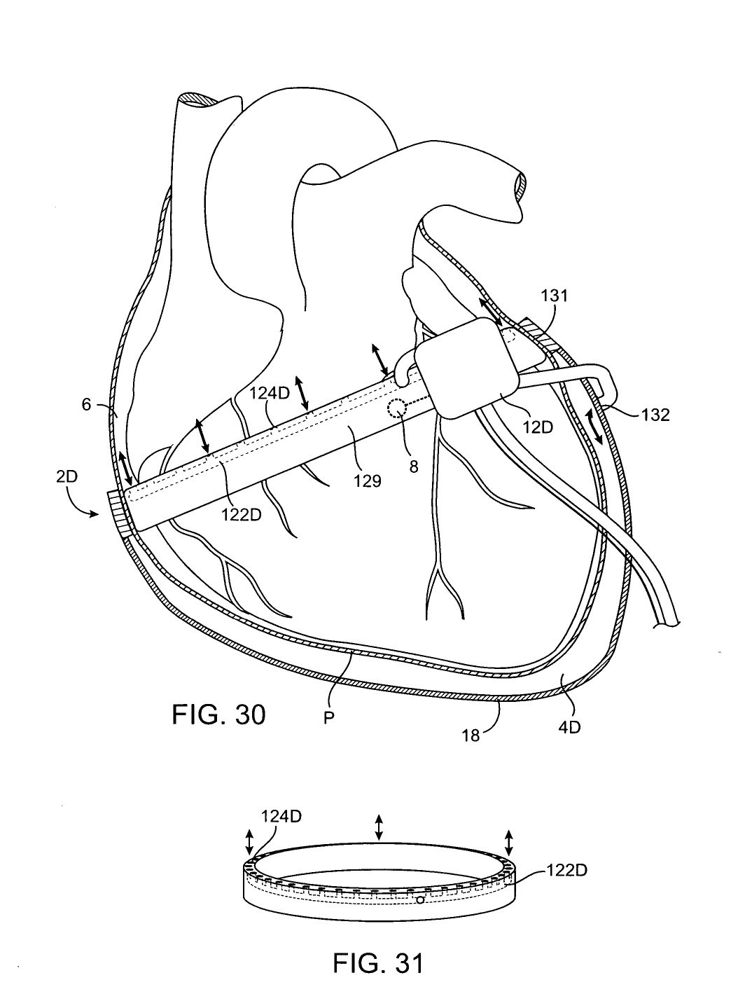

Referring to FIGS. 30 and 31, still another cardiac assist device 2D is shown wherein the same or similar reference numbers refer to the same or similar structure. The cardiac assist device 2D has a body 129 positioned between the pericardium and epicardium and a jacket 18 positioned outside the pericardium, rather than inside the pericardium as disclosed in other embodiments described herein, so that a first pumping space 4D is created between the jacket 18 and the exterior surface of the pericardium. A pump 12D delivers fluid to the pumping space 4 through a fluid outlet 132. The pump 12D also delivers fluid to a fluid distribution channel 122D which distributes fluid to fluid outlets 124D for the second pumping space 6. When fluid is pumped into the pumping space 4D, the fluid pressure in the pumping space 4 forces the pericardium into contact with the epicardium to aid the heart in pumping blood. The jacket 18 has a sealing surface 131 which is sealed to and encircles the exterior surface of the pericardium to seal the pumping space in any manner described herein or any other suitable manner. The cardiac assist device 2D also includes a second pumping space 6 which assist one or both atria. The second pumping space 6 is created in the natural space between the epicardium and pericardium. Of course, the jacket 18 and/or sheath may be used in the pumping spaces 4D, 6 without departing from the scope of the invention.

Although the present invention has been described in connection with various specific embodiments it is understood that various modifications of the present invention may be undertaken without departing from the scope of the invention. Furthermore, any disclosure related to one of the pumping spaces is expressly incorporated for use in the other pumping space.

1-10. (canceled)

11. A method of assisting the heart, comprising the steps of:
providing a cardiac assist device including a fluid delivery system having a pump;
creating a first pumping space and a second pumping space, the first pumping space being positioned between the pericardium and the ventricular epicardium, the second pumping space being positioned between the pericardium and the atrial epicardium;
monitoring cardiac activity;
pumping fluid into and out of the first pumping space, the fluid being pumped into the first pumping space to assist ventricular pumping of blood; and
pumping fluid into and out of the second pumping space, the fluid being pumped into the second pumping space to assist atrial pumping of blood.

12. The method of claim 11, wherein:
the pumping steps are carried out with the fluid being pumped between the first and second pumping spaces.

13. The method of claim 11, wherein:
the pumping step is carried out with the fluid in at least one of the first and second pumping spaces being in direct contact with the epicardium.

14. The method of claim 11, wherein;
the providing step is carried out with at least one of the first and second pumping spaces being a closed space which does not permit the fluid to contact the epicardium.

15. The method of claim 11, further comprising the step of:
forming a seal between the cardiac assist device and the epicardium which encircles the heart.

16-49. (canceled)

50. A system for assisting the heart, comprising:
a body;
a fluid delivery system coupled to the body, the fluid delivery system including a pump;
a first fluid delivery lumen coupled to the fluid delivery system, the first fluid delivery lumen delivering fluid from the pump to assist at least one ventricle in pumping blood; and
a second fluid delivery lumen coupled to the fluid delivery system, the second fluid delivery lumen delivering fluid from the fluid delivery system to assist at least one atrium in pumping blood;
a cardiac monitoring device which monitors cardiac activity, the cardiac monitoring device being operably coupled to the fluid delivery system so that fluid is delivered through the first and second fluid delivery lumens to assist at least one ventricle and at least one atrium, respectively, in response to the cardiac activity measured by the cardiac monitoring device; and
a control system coupled to the cardiac monitoring system and operably coupled to the fluid delivery system, the control system receiving information from the cardiac monitoring system and controlling the fluid delivery system to deliver fluid through the first delivery lumen in a manner which assists the at least one ventricle in pumping blood, the control system also controlling the fluid delivery system to deliver fluid through the second delivery lumen in a manner which assist the at least one atrium in pumping blood.

51. The system of claim 50, wherein:
the fluid delivery system delivers the same fluid through the first and second delivery lumens.

52. The system of claim 50, wherein:
the body includes a first sealing surface on an inner side, the first sealing surface forming a closed loop which encircles the heart when the body is positioned around the heart, the first sealing surface forming a seal with the epicardium which encircles the heart when the body is positioned around the heart, the body also including a second sealing surface on an outer side, the second sealing surface also forming a closed loop, the second sealing surface forming a seal with the pericardium when the body is positioned around the heart, the body creating a first pumping space which receives fluid from the first delivery lumen and a second pumping space which receives fluid from the second delivery lumen.

53. The system of claim 52, wherein:
the fluid delivery system delivers fluid through the first delivery lumen when withdrawing fluid from the second pumping space and delivering fluid through the second delivery lumen when withdrawing fluid from the first pumping space.

54. The system of claim 50, wherein:
the fluid delivery system is configured to deliver fluid in direct contact with the epicardium when the body is positioned adjacent to at least a portion of the heart.

55. The system of claim 50, further comprising:
a chamber coupled to at least one of the fluid delivery lumens, the chamber being a closed chamber which does not permit the fluid to come into direct contact with the epicardium.

56-320. (canceled)

321. The system of claim 50, wherein:
the fluid delivery system includes a reservoir which is fluidly coupled to the fluid delivery system.

322. The system of claim 50, wherein:
the fluid delivery system is configured to withdraw fluid from the first fluid delivery lumen when a failure condition exists.

323. The system of claim 322, wherein:
the control system includes a pressure sensor which monitors fluid pressure, the failure condition being an unacceptable fluid pressure.

324. The system of claim 50, wherein:
the fluid control system is configured to deliver fluid into the first lumen during at least a portion of systole and withdrawing fluid from the first lumen during at least a portion of diastole.

325. The system of claim 50, wherein:
the first fluid delivery lumen is coupled to a first pumping chamber configured to fit adjacent to at least a portion of the ventricle.

326. The system of claim 325, wherein:
the first pumping chamber is configured to apply external pressure to the ventricle when fluid is delivered to the first pumping chamber.

327. The system of claim 326, further comprising:
a sheath which forms part of the first pumping chamber, the sheath being flexible and compliant so that the sheath conforms to the natural anatomy.

328. The system of claim 50, wherein:
the second fluid delivery lumen is coupled to a second pumping chamber configured to fit adjacent to at least a portion of the atrium.

329. The system of claim 328, wherein:
the second pumping chamber is configured to apply external pressure to the atrium when fluid is delivered to the second pumping chamber.

330. The system of claim 50, wherein:
the pump is a unidirectional pump having at least one valve which is used to reverse the fluid flow direction.

331. The system of claim 50, wherein:
the pump is a bi-directional pump, wherein the pump directs fluid to the first fluid delivery lumen in one flow direction and to the second fluid delivery lumen in the other flow direction.

332. The system of claim 50, further comprising:
a third fluid delivery lumen coupled to the fluid delivery system, the third fluid delivery lumen delivering fluid from the pump to assist the other ventricle in pumping blood.

333-346. (canceled)
